(19) United States
(12) Patent Application Publication
(10) Pub. No.: US 2025/0284760 A1
(43) Pub. Date: Sep. 11, 2025

(54) DELAYED JUST-IN-TIME MODEL UPDATE USING BLOCKWISE RECURSIVE PARTIAL LEAST SQUARES (PLS) FOR SLOW AND FAST INTERACTIVE PROCESSES

(71) Applicant: Yokogawa Electric Corporation, Tokyo (JP)

(72) Inventors: Venkata krishnaprasad Reddy VURUBINDI, Atmakuru (IN); Sanjay VENUGOPAL, Bengaluru (IN); Srinivas VENTRAPRAGADA, Bengaluru (IN); Kumar SATYAM, Bettiah (IN); Arunkumar KANNAN, Bengaluru (IN)

(21) Appl. No.: 18/601,703

(22) Filed: Mar. 11, 2024

(51) Int. Cl. G06F 17/11

(52) U.S. Cl. CPC G06F 17/11

(57) ABSTRACT

Most chemical processes are nonlinear in nature and are time-varying in nature. Some processes are fast changing and some are slow changing in nature. As the operating conditions change, it is important that the model and its parameters be updated to realize the benefits of model-based control. Systems and methods are provided for updating a model, the method including detecting a change in an output of the model responsive to providing a set of inputs to the model, in response to detecting the change in the output, triggering a new model detection flag using a blockwise recursive partial least squares (RPLS) algorithm. The model is further strengthened by using a delayed just-in-time update.

### FIELD OF THE DISCLOSURE

The invention relates generally to systems and methods for modeling processes and particularly to modeling non-linear chemical processes with noisy inputs.

### BACKGROUND

Incorporated herein by reference is the document by P. Vijaysai, R. D. Gudi, and S. Lakshminarayanan, titled, “Identification on Demand Using a Blockwise Recursive Partial Least-Squares Technique,” in Ind. Eng. Chem. Res. 2003, 42, 540-554 (Publication ie020042r) [online]; Mumbai, India: Indian Institute of Technology; and Singapore, Singapore: National University of Singapore; publication date: Jan. 4, 2003; American Chemical Society [retrieved 2023 Dec. 11]; retrieved from the Internet: <URL:doi.org/10.1021/ie020042r>.

Various methodologies for modeling real-world processes are utilized in various industries, such as chemical processes. For example, a crude oil refinery may utilize a particular model to plan for, and execute, a particular mode of operation, such as to maximize a particular output (e.g., a particular petroleum product, waste product, etc.) and/or minimize a particular input (e.g., feed stock, additive, catalyst, energy input, etc.), which may further be complicated by environmental inputs (e.g., change in ambient temperature). Changes to an input, including environmental inputs or feed characteristics, may produce linear or nonlinear changes to the outputs. Real-world chemical processes are commonly nonlinear and challenging to model.

Modeling commonly uses recursive least squares (RLS) and ordinary least squares (OLS) with their computational deficiencies modified with ill-conditioned matrices, data with inferior quality, and the highly correlated inputs. Partial least squares (PLS) algorithms are well-known mythologies for correlated and noisy data.

Despite the modeling solutions available in the prior art, problems remain.

### SUMMARY

Modeling a process, such as a chemical process that is changing or may change (e.g., a change in an input, etc.), requires determining whether or not a new model update is needed. In particular, determining the changes in the data are sufficient to warrant the need for an updated model. For example, determining how much of a change in the data, and to which data, is sufficient to justify the use of a new model and/or the termination of an existing model and is outside the teachings of the prior art.

These and other needs are addressed by the various embodiments and configurations of the present invention. The present invention can provide a number of advantages depending on the particular configuration. These and other advantages will be apparent from the disclosure of the invention(s) contained herein.

In one embodiment, a blockwise recursive partial least squares (PLS) technique is disclosed. This technique utilizes a condition number and forgetting factor, which overcome the shortcomings of the prior art.

In another embodiment, limitations in a just-in-time model are overcome by use of a delayed just-in-time model. A just-in-time model using simulated, by representative of real-world data, of complex relationships, some of which changed faster than others, with a change in the steady state gain and as well as the dynamics can be further improved by a delayed just-in-time model.

In some aspects, the techniques described herein relate to a method of updating a model, the method including: detecting a change in an output of the model responsive to providing a set of inputs to the model; in response to detecting the change in the output, triggering a new model detection flag using a blockwise recursive partial least squares (RPLS) algorithm; assessing a performance of a new model using a set of prediction metrics; storing the new model in memory; comparing a performance of the model using the set of inputs with the performance of the new model using the set of prediction metrics; and based on the comparison and in response to the comparison indicating that the performance of the new model using the set of prediction metrics is improved relative to the performance of the model using the set of inputs, replacing the model with the new model in run-time.

In some aspects, the techniques described herein relate to a method, wherein the memory includes buffer memory.

In some aspects, the techniques described herein relate to a method, further including: validating the new model on buffer process data.

In some aspects, the techniques described herein relate to a method, wherein the buffer process data includes at least one of the following: a past data block, a present data block, and a data block to be received.

In some aspects, the techniques described herein relate to a method, wherein a size of the buffer memory is selected to minimize an impact on data processing while validating the new model.

In some aspects, the techniques described herein relate to a method, further including: iteratively repeating the detecting, the triggering, the assessing, the storing, the comparing, and the replacing.

In some aspects, the techniques described herein relate to a method, further including: storing the model in memory with a data signature that is unique to the model; and enabling retrieval of the model in real-time based on a detection of another data signature in process data having a predetermined similarity to the data signature of the model stored in memory.

In some aspects, the techniques described herein relate to a method, further including: maintaining a model library that includes the model and the new model, wherein the model library is indexed according to data signatures associated with each model in the model library.

In some aspects, the techniques described herein relate to a method, further including: determining an angle between weight matrices of a master model and weight matrices of the new model; and correlating the angle to a measure of change between an input block and output block of the master model and the new model.

In some aspects, the techniques described herein relate to a system, including: a processor; and a memory capable of storing data thereon that, when processed by the processor, cause the processor to: detect a change in an output of a model responsive to providing a set of inputs to the model; in response to detecting the change in the output, trigger a new model detection flag using a blockwise recursive partial least squares (RPLS) algorithm; assess a performance of a new model using a set of prediction metrics; store the new model in a buffer; compare a performance of the model using the set of inputs with the performance of the new model using the set of prediction metrics; and based on the comparison and in response to the comparison indicating that the performance of the new model using the set of prediction metrics is improved relative to the performance of the model using the set of inputs, replace the model with the new model in run-time.

In some aspects, the techniques described herein relate to a system, wherein the data further enables the processor to: validate the new model on buffer process data.

In some aspects, the techniques described herein relate to a system, wherein the buffer process data includes at least one of the following: a past data block, a present data block, and a data block to be received.

In some aspects, the techniques described herein relate to a system, wherein a size of the buffer memory is selected to minimize an impact on data processing while validating the new model.

In some aspects, the techniques described herein relate to a system, wherein the data further enables the processor to: iteratively repeat the detecting, the triggering, the assessing, the storing, the comparing, and the replacing.

In some aspects, the techniques described herein relate to a system, wherein the data further enables the processor to: store the model in memory with a data signature that is unique to the model; and enable retrieval of the model in real-time based on a detection of another data signature in process data having a predetermined similarity to the data signature of the model stored in memory.

In some aspects, the techniques described herein relate to a system, wherein the data further enables the processor to: maintain a model library that includes the model and the new model, wherein the model library is indexed according to data signatures associated with each model in the model library.

In some aspects, the techniques described herein relate to a system, wherein the data further enables the processor to: determine an angle between weight matrices of a master model and weight matrices of the new model; correlate the angle to a measure of change between an input block and output block of the master model and the new model.

In some aspects, the techniques described herein relate to a non-transitory computer-readable medium including processor-executable instructions stored thereon that enable a processor to: detect a change in an output of a model responsive to providing a set of inputs to the model; in response to detecting the change in the output, trigger a new model detection flag using a blockwise recursive partial least squares (RPLS) algorithm; assess a performance of a new model using a set of prediction metrics; store the new model in a buffer; compare a performance of the model using the set of inputs with the performance of the new model using the set of prediction metrics; and based on the comparison and in response to the comparison indicating that the performance of the new model using the set of prediction metrics is improved relative to the performance of the model using the set of inputs, replace the model with the new model in run-time.

In some aspects, the techniques described herein relate to a non-transitory computer-readable medium, wherein the instructions further enable the processor to: validate the new model on buffer process data, wherein the buffer process data includes at least one of the following: a past data block, a present data block, and a data block to be received.

In some aspects, the techniques described herein relate to a non-transitory computer-readable medium, wherein the instructions further enable the processor to determine an angle between weight matrices of a master model and weight matrices of the new model, and correlate the angle to a measure of change between an input block and output block of the master model and the new model.

A system on a chip (SoC) including any one or more of the above aspects or aspects of the embodiments described herein.

One or more means for performing any one or more of the above or aspects of the embodiments described herein.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

Any of the above aspects or aspects of the embodiments described herein, wherein the data storage comprises a non-transitory storage device, which may further comprise at least one of: an on-chip memory within the processor, a register of the processor, an on-board memory co-located on a processing board with the processor, a memory accessible to the processor via a bus, a magnetic media, an optical media, a solid-state media, an input-output buffer, a memory of an input-output component in communication with the processor, a network communication buffer, and a networked component in communication with the processor via a network interface.

It is to be appreciated that any feature described herein can be claimed in combination with any other feature(s) as described herein, regardless of whether the features come from the same described embodiment.

The phrases “at least one,” “one or more,” “or,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B, and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible, non-transitory medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably and include any type of methodology, process, mathematical operation or technique.

The term “means” as used herein shall be given its broadest possible interpretation in accordance with 35 U.S.C., Section 112(f) and/or Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The preceding is a simplified summary of the invention to provide an understanding of some aspects of the invention. This summary is neither an extensive nor exhaustive overview of the invention and its various embodiments. It is intended neither to identify key or critical elements of the invention nor to delineate the scope of the invention but to present selected concepts of the invention in a simplified form as an introduction to the more detailed description presented below. As will be appreciated, other embodiments of the invention are possible utilizing, alone or in combination, one or more of the features set forth above or described in detail below. Also, while the disclosure is presented in terms of exemplary embodiments, it should be appreciated that an individual aspect of the disclosure can be separately claimed.

### BRIEF DESCRIPTION OF THE DRAWINGS

The present disclosure is described in conjunction with the appended figures:

FIG. 1 depicts a process in accordance with embodiments of the present disclosure;

FIGS. 2A and 2B depict a process in accordance with embodiments of the present disclosure;

FIG. 3 depicts memories with contents maintained therein in accordance with embodiments of the present disclosure; and

FIG. 4 depicts a system in accordance with embodiments of the present disclosure.

### DETAILED DESCRIPTION

The ensuing description provides embodiments only and is not intended to limit the scope, applicability, or configuration of the claims. Rather, the ensuing description will provide those skilled in the art with an enabling description for implementing the embodiments. It will be understood that various changes may be made in the function and arrangement of elements without departing from the spirit and scope of the appended claims.

Any reference in the description comprising a numeric reference number, without an alphabetic sub-reference identifier when a sub-reference identifier exists in the figures, when used in the plural, is a reference to any two or more elements with the like reference number. When such a reference is made in the singular form, but without identification of the sub-reference identifier, it is a reference to one of the like numbered elements, but without limitation as to the particular one of the elements being referenced. Any explicit usage herein to the contrary or providing further qualification or identification shall take precedence.

The exemplary systems and methods of this disclosure will also be described in relation to analysis software, modules, and associated analysis hardware. However, to avoid unnecessarily obscuring the present disclosure, the following description omits well-known structures, components, and devices, which may be omitted from or shown in a simplified form in the figures or otherwise summarized.

For purposes of explanation, numerous details are set forth in order to provide a thorough understanding of the present disclosure. It should be appreciated, however, that the present disclosure may be practiced in a variety of ways beyond the specific details set forth herein.

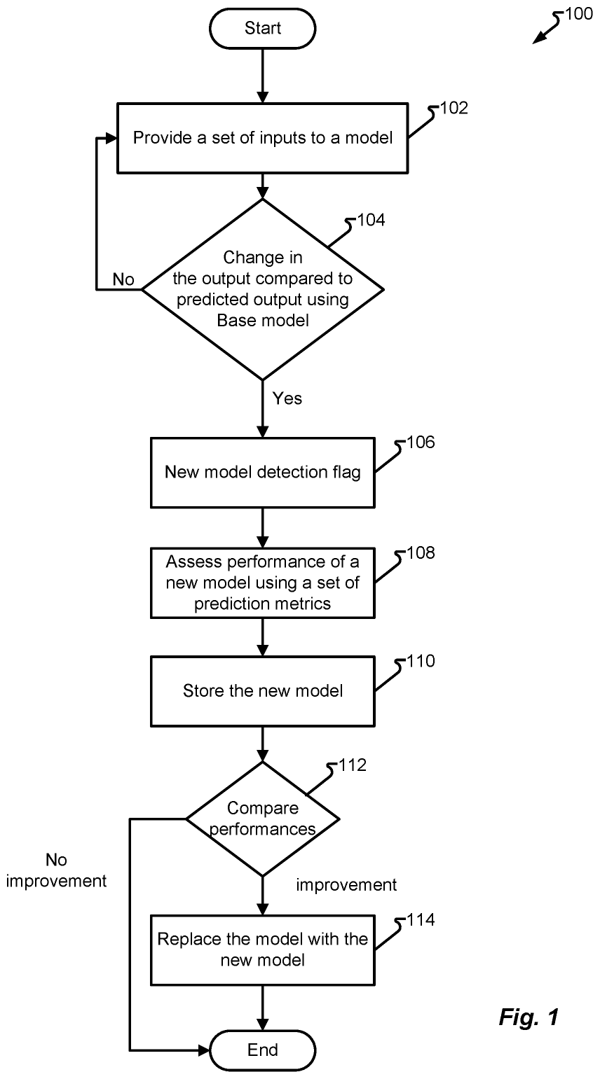

FIG. 1 depicts process 100 in accordance with embodiments of the present disclosure. In one embodiment, process 100 is embodied as machine-readable instructions maintained in a non-transitory memory that when read by a machine, such by one or more processors of a server or servers, cause the machine to execute the instructions and thereby execute process 100.

Process 100 begins and, in step 102, a set of inputs is provided to a model of a real-world process, such as a chemical process. Test 104 determines whether there is a change in the output of the model compared to measured output value from the chemical process. If test 104 is determined in the negative, process 100 may loop back to step 102 to provide another set of inputs to the model or, optionally, terminate. The looping of step 102 and test 104 may continue indefinitely or until terminated.

If test 104 is determined in the affirmative, step 106 triggers a new model detection flag, such as to set a value maintained in a data storage or computer memory, such as a buffer memory. Step 106 may utilize a blockwise recursive partial least squares (RPLS) algorithm to trigger the new model detection flag. Next, step 108 assesses the performance of a new model using a set of prediction metrics and, in step 110, stores the new model in a memory or other data storage. The set of prediction metrics include but are not limited to R², adjusted R², RMSE (Root Mean Squared Error) in some embodiments of the present invention. Test 112 then compares the performances of the model, using the set of inputs, with the performance of the new model using the set of prediction metrics to determine if there is an improvement relative to the performance of the model using the set of inputs. If test 112 determines an improvement resulted, step 114 replaces the model with the new model. The replacement in step 114 may occur in run-time. If test 112 determines that there is no improvement, then no replacement of the model is performed. Process 100 may then end. Optionally, process 100 may loop back to step 102 and repeat.

Optionally, the new model is validated on buffer process data, which may further comprise at least one of a past data block, a present data block, and a data block to be received. The size of the buffer memory may be selected to minimize the impact of a computational burden placed on a process while validating the new model.

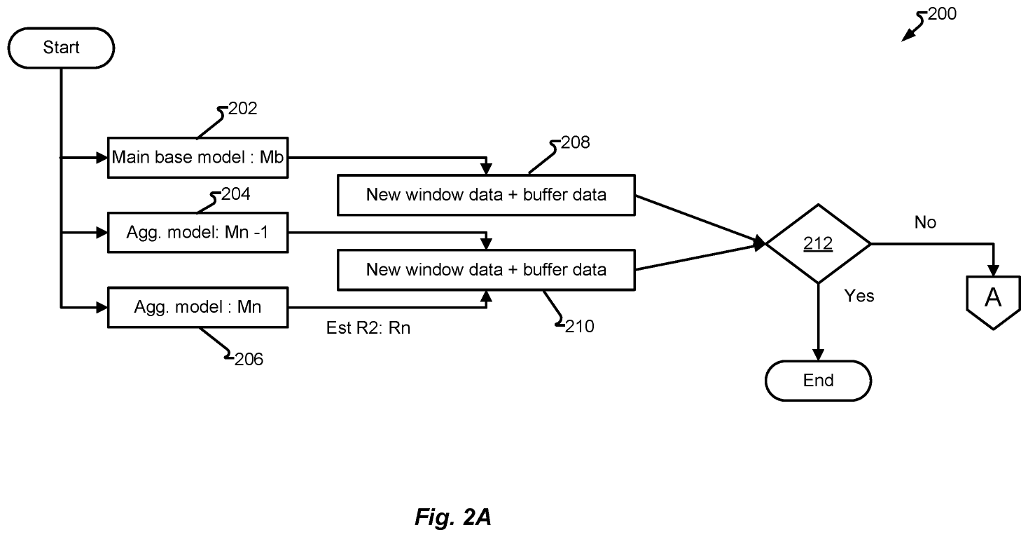

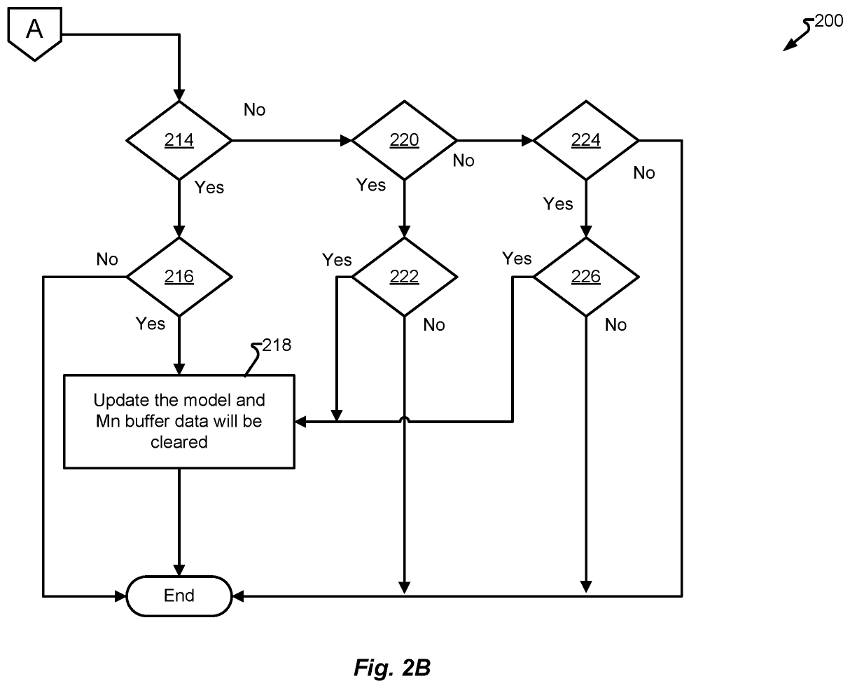

FIGS. 2A and 2B depict process 200 in accordance with embodiments of the present disclosure. In one embodiment, process 200 is embodied as machine-readable instructions maintained in a non-transitory memory that when read by a machine, such by one or more processors of a server or servers, cause the machine to execute the instructions and thereby execute process 200.

In one embodiment, a processor(s) is provided coupled to a buffer or other computer memory. The buffer comprises a number of data blocks. The data blocks may include a past data block (“n−1”), a present data block (“n”), and a future data block (“n+1”).

In process 200, MCT is the model confidence threshold refers to a threshold value above which the model is considered reliable or the output is acceptable, such as 0.6; RMSE (Root Mean Squared Error) % is the RMSE percentage improvement which refers to the percentage root means square error between a new aggregated model and a previous model, such as 80; R²T (R squared threshold) is the R² improvement threshold between the current and previous models, if R²T is favorable, the new model will be chosen. and may have a value such as 0.40.

In one embodiment, a system executing process 200 detects a change in output response to a set of inputs and, as a result, a new model detection flag is triggered. The system triggers identification of a new model using the blockwise RPLS technique. The new model identified may be stored in a memory and tested with a next set of blocks. The data may comprise user configurable data to allow the user to see the degree of fit to the model. If the fit satisfies the new model update criterion, then the main model is updated with the new model information, such as supplemental model information.

In one embodiment, process 200 begins and step 202 wherein a main base model (“M_{b}”) is accessed and applied to data 208 comprising new window data and buffer data. In an embodiment, the base model is used to establish relationship between a set of input variables and output variables. The base model is used to predict the output variables at any time using predetermined set of input variables. In another embodiment, process 200 begins at step 204 accessing a previous model (“Mn−1”) and at step 206 a current model (“Mn”) is applied to data 210, comprising new window data and buffer data. An estimated “R²” for current model (Mn) is “R²n”. 204 and 206 use Aggregated data and models. Aggregated data means at least one of a past data block, a present data block, and a data block to be received. Aggregated model means a model prepared using the aggregated data.

Next, test 212 determines if (a) Mn−1 r2>MCT and (b) Mn r2<MCT. If test 212 is determined in the affirmative, process 200 ends as there is no need to update the model. The window data will be added to the buffer data (Mn−1) and will be available for subsequent use. The window data refers to a predefined amount of data to be used. If test 212 is determined in the negative, test 214 (via off-page connector “A”) determines if Mb r2==Mn r2. If test 214 is determined in the affirmative, test 216 then determines if (a) Mn r2>R2T and (b) Mn RMSE has improved by RMSET %. If test 214 is determined in the negative, processing continues to test 220.

Test 220 determines if (a) Mb r2>Mn r2 and (b) Mb rc>Mn−1 r2. If test 220 is determined in the affirmative, processing continues to test 222. If test 220 is determined in the negative, processing continues to test 224. Test 222 determines if (a) Mb r2>R2T and (b) Mb RMSE has improved by RMSET % which refers to a percent change in RMSE. If test 222 is determined in the negative, processing may end. If test 222 is determined in the affirmative, processing continues to step 218. In step 218 the model is updated the Mn buffer is cleared.

Test 224 determines if (a) Mn r2>Mn−1 r2 and (b) Mn r2>Mb 42. If test 224 is determined in the negative, processing may end. If test 224 is determined in the affirmative, processing continues to test 226. Test 226 determines if (a) Mn r2>R2T and (b) Mn RMSE has improved by RMSET %. If test 226 is determined in the negative, processing may end. If test 226 is determined in the affirmative, processing continues to step 218.

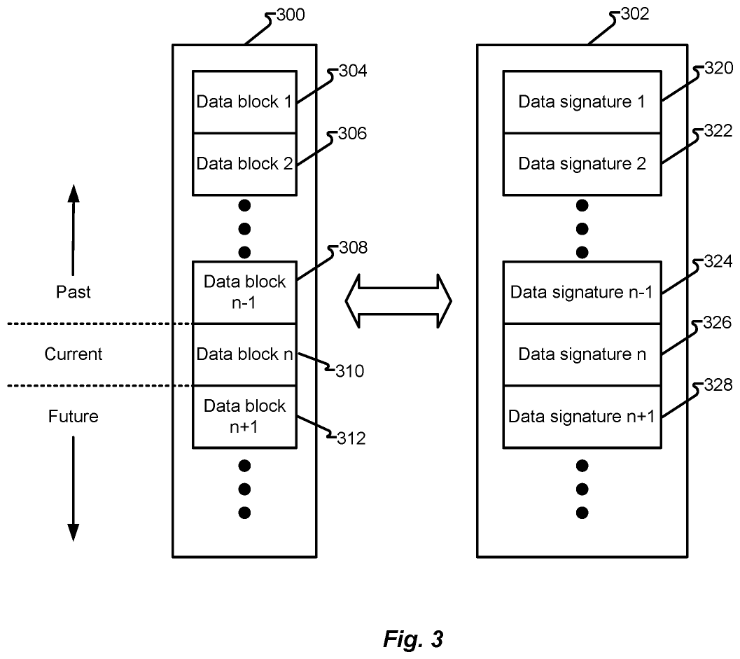

FIG. 3 illustrates memory 300 and memory 302 in accordance with embodiments of the present disclosure. Memory 300 and/or memory 302 may be embodied as a computer memory, data storage, distributed (e.g., “cloud”) storage, or other computer data storage. Memory 300 and memory 302 may be discrete data storage components or different portions of the same data storage components.

Memory 300 comprises a number of data blocks. It should be appreciated by those of ordinary skill in the art that memory 300 may comprise more or fewer data blocks than those illustrated herein. In one embodiment, memory 300 comprises a number of data blocks associated with past data, such as data block 1 (304), data block 2 (306), and data block n−1 (308). As mentioned above, the number of past data blocks may be fewer than those illustrated or greater, such as additional data blocks between data block 2 (306) and data block n−1 (308).

Memory 300 comprises a current data block, such as data block n (310). Memory 300 may comprise one or more additional future data blocks, such as data block n+1 (312).

Memory 300, or portions thereof, may be embodied as a model library maintaining a collection of data blocks. Additionally or alternatively, memory 300 may comprise the model (see FIGS. 2A and 2B) and/or the new model (see Id.) comprise a library of data blocks. As a further option, the model and/or the new model may be indexed based on a corresponding data signature, such as may be maintained in memory 302. As yet another option, memory 300 may maintain a number of new models, and memory 302 may maintain a corresponding data signature for each of the number of new models.

Memory 302 comprises a data signature corresponding to the data blocks maintained in memory 302. The data signatures uniquely identify the corresponding data block. For example, data signature 1 (320) corresponds to data block 1 (304); data signature 2 (322) corresponds to data block 2 (306); data signature n−1 (324) corresponds to data block n−1 (308); data signature n (326) corresponds to data block n (310); and data signature n+1 (328) corresponds to data block n+1 (312). The inclusion or omission of other data blocks from memory 300 may correspond to a similar inclusion or omission of other data signatures from memory 302. Additionally or alternatively, as introduced above, memory 302 may comprise data signatures for the model and/or new model. As a result, the model, new model, and/or data blocks may be indexed by the data signatures maintained in memory 302. The data signatures are unique and the data signature corresponding to the model uniquely identifies the model. Similarly, the data signature corresponding to the new model uniquely identifies the new model.

In another embodiment, the model is stored in memory 300 is associated with a particular data signature and may have a data signature with a known pattern. As a result, a model may be retrieved in real-time based on a detection of another data signature in processing data having a predetermined similarity to the data signature of the model stored in a memory, such as memory 302. As a further option, a forgetting factor may be applied to avoid “blow ups.” Wherein the covariance matrix will grow with insufficient values in the matrix due to the lack of information in the new data and may lead to exponential growth of covariance matrix if past data was continuously discounted. The forgetting factor is a value that is used to control the rate at which older data is “forgotten” when updating a model's parameters. The forgetting factor determines how much weight is given to older data points compared to more recent data points when updating the model. In an embodiment, a value between 0 and 1, where a higher value gives more weight to older data and a lower value gives more weight to more recent data.

In another embodiment, a master model is maintained in data storage, such as memory 300 and/or memory 302. An angle between weighted matrices of the master model and weighted matrices of the new model are determined. The extent of information to be discounted is estimated as the change in the angle between the first vector of the weight matrices of the master model and the new model.

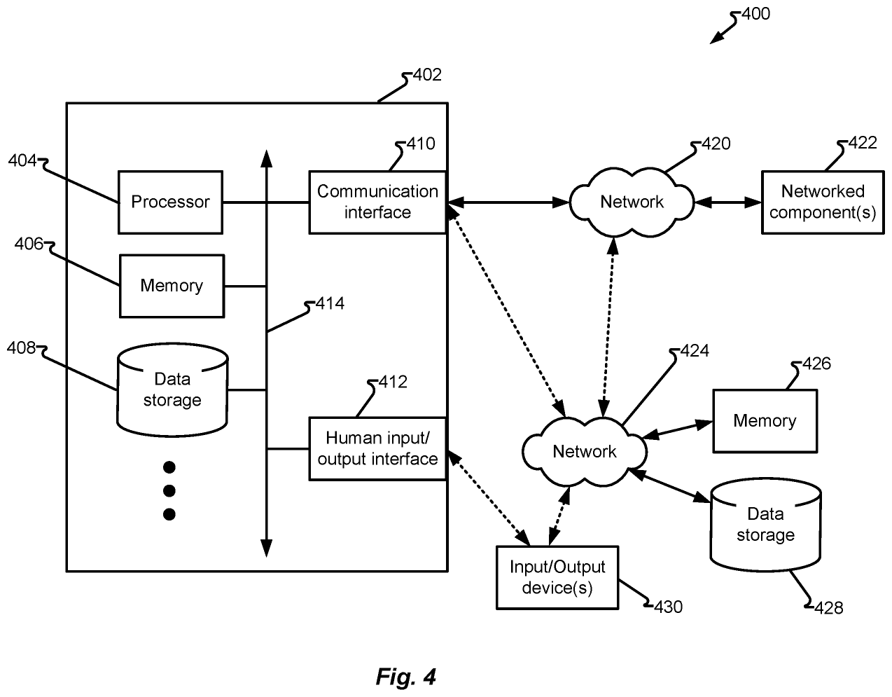

FIG. 4 depicts device 402 in system 400 in accordance with embodiments of the present disclosure. In one embodiment, device 402 may be embodied, in whole or in part, as a single device comprising various components and connections to other components and/or systems. Or as an organized plurality of devices, each performing a portion of the computing, communication, and/or data storage tasks. The components are variously embodied and may comprise processor 404. The term “processor,” as used herein, refers exclusively to electronic hardware components comprising electrical circuitry with connections (e.g., pin-outs) to convey encoded electrical signals to and from the electrical circuitry. Processor 404 may comprise programmable logic functionality, such as determined, at least in part, from accessing machine-readable instructions maintained in a non-transitory data storage, which may be embodied as circuitry, on-chip read-only memory, computer memory 406, data storage 408, etc., that cause the processor 404 to perform the steps of the instructions. For example, processor 404 may perform the instructions to execute process 100, process 200, and/or other processes.

In one embodiment, memory 300 and/or memory 302 are embodied as computer memory 406. In another embodiment, memory 300 and/or memory 302 may be embodied as one or more of data storage 408, memory 426, and/or data storage 428.

Processor 404 may be further embodied as a single electronic microprocessor or multiprocessor device (e.g., multicore) having electrical circuitry therein which may further comprise a control unit(s), input/output unit(s), arithmetic logic unit(s), register(s), primary memory, and/or other components that access information (e.g., data, instructions, etc.), such as received via bus 414, executes instructions, and outputs data, again such as via bus 414. In other embodiments, processor 404 may comprise a shared processing device that may be utilized by other processes and/or process owners, such as in a processing array within a system (e.g., blade, multi-processor board, etc.) or distributed processing system (e.g., “cloud”, farm, etc.). It should be appreciated that processor 404 is a non-transitory computing device (e.g., electronic machine comprising circuitry and connections to communicate with other components and devices). Processor 404 may operate a virtual processor, such as to process machine instructions not native to the processor (e.g., translate the VAX operating system and VAX machine instruction code set into Intel® 9xx chipset code to enable VAX-specific applications to execute on a virtual VAX processor). However, as those of ordinary skill understand, such virtual processors are applications executed by hardware, more specifically, the underlying electrical circuitry and other hardware of the processor (e.g., processor 404). Processor 404 may be executed by virtual processors, such as when applications (i.e., Pod) are orchestrated by Kubernetes. Virtual processors enable an application to be presented with what appears to be a static and/or dedicated processor executing the instructions of the application, while underlying non-virtual processor(s) are executing the instructions and may be dynamic and/or split among a number of processors.

In addition to the components of processor 404, device 402 may utilize computer memory 406 and/or data storage 408 for the storage of accessible data, such as instructions, values, etc. Communication interface 410 facilitates communication with components, such as processor 404 via bus 414 with components not accessible via bus 414 and may be embodied as a network interface (e.g., ethernet card, wireless networking components, USB port, etc.). Communication interface 410 may be embodied as a network port, card, cable, or other configured hardware device. Additionally or alternatively, human input/output interface 412 connects to one or more interface components to receive and/or present information (e.g., instructions, data, values, etc.) to and/or from a human and/or electronic device. Examples of input/output devices 430 that may be connected to input/output interface include, but are not limited to, keyboard, mouse, trackball, printers, displays, sensor, switch, relay, speaker, microphone, still and/or video camera, etc. In another embodiment, communication interface 410 may comprise, or be comprised by, human input/output interface 412. Communication interface 410 may be configured to communicate directly with a networked component or configured to utilize one or more networks, such as network 420 and/or network 424.

Network 420 may be a wired network (e.g., Ethernet), wireless (e.g., WiFi, Bluetooth, cellular, etc.) network, or combination thereof and enable device 402 to communicate with networked component(s) 422. In other embodiments, network 420 may be embodied, in whole or in part, as a telephony network (e.g., public switched telephone network (PSTN), private branch exchange (PBX), cellular telephony network, etc.).

Additionally or alternatively, one or more other networks may be utilized. For example, network 424 may represent a second network, which may facilitate communication with components utilized by device 402. For example, network 424 may be an internal network to a business entity or other organization, whereby components are trusted (or at least more so) than networked components 422, which may be connected to network 420 comprising a public network (e.g., Internet) that may not be as trusted.

Components attached to network 424 may include computer memory 426, data storage 428, input/output device(s) 430, and/or other components that may be accessible to processor 404. For example, computer memory 426 and/or data storage 428 may supplement or supplant computer memory 406 and/or data storage 408 entirely or for a particular task or purpose. As another example, computer memory 426 and/or data storage 428 may be an external data repository (e.g., server farm, array, “cloud,” etc.) and enable device 402, and/or other devices, to access data thereon. Similarly, input/output device(s) 430 may be accessed by processor 404 via human input/output interface 412 and/or via communication interface 410 either directly, via network 424, via network 420 alone (not shown), or via networks 424 and 420. Each of computer memory 406, data storage 408, computer memory 426, data storage 428 comprise a non-transitory data storage comprising a data storage device.

It should be appreciated that computer readable data may be sent, received, stored, processed, and presented by a variety of components. It should also be appreciated that components illustrated may control other components, whether illustrated herein or otherwise. For example, one input/output device 430 may be a router, a switch, a port, or other communication component such that a particular output of processor 404 enables (or disables) input/output device 430, which may be associated with network 420 and/or network 424, to allow (or disallow) communications between two or more nodes on network 420 and/or network 424. One of ordinary skill in the art will appreciate that other communication equipment may be utilized, in addition or as an alternative, to those described herein without departing from the scope of the embodiments.

In the foregoing description, for the purposes of illustration, methods were described in a particular order. It should be appreciated that in alternate embodiments, the methods may be performed in a different order than that described without departing from the scope of the embodiments. It should also be appreciated that the methods described above may be performed as algorithms executed by hardware components (e.g., circuitry) purpose-built to carry out one or more algorithms or portions thereof described herein. In another embodiment, the hardware component may comprise a general-purpose microprocessor (e.g., CPU, GPU) that is first converted to a special-purpose microprocessor. The special-purpose microprocessor then having had loaded therein encoded signals causing the, now special-purpose, microprocessor to maintain machine-readable instructions to enable the microprocessor to read and execute the machine-readable set of instructions derived from the algorithms and/or other instructions described herein. The machine-readable instructions utilized to execute the algorithm(s), or portions thereof, are not unlimited but utilize a finite set of instructions known to the microprocessor. The machine-readable instructions may be encoded in the microprocessor as signals or values in signal-producing components by, in one or more embodiments, voltages in memory circuits, configuration of switching circuits, and/or by selective use of particular logic gate circuits. Additionally or alternatively, the machine-readable instructions may be accessible to the microprocessor and encoded in a media or device as magnetic fields, voltage values, charge values, reflective/non-reflective portions, and/or physical indicia.

In another embodiment, the microprocessor further comprises one or more of a single microprocessor, a multi-core processor, a plurality of microprocessors, a distributed processing system (e.g., array(s), blade(s), server farm(s), “cloud”, multi-purpose processor array(s), cluster(s), etc.) and/or may be co-located with a microprocessor performing other processing operations. Any one or more microprocessors may be integrated into a single processing appliance (e.g., computer, server, blade, etc.) or located entirely, or in part, in a discrete component and connected via a communications link (e.g., bus, network, backplane, etc. or a plurality thereof).

Examples of general-purpose microprocessors may comprise, a central processing unit (CPU) with data values encoded in an instruction register (or other circuitry maintaining instructions) or data values comprising memory locations, which in turn comprise values utilized as instructions. The memory locations may further comprise a memory location that is external to the CPU. Such CPU-external components may be embodied as one or more of a field-programmable gate array (FPGA), read-only memory (ROM), programmable read-only memory (PROM), erasable programmable read-only memory (EPROM), random access memory (RAM), bus-accessible storage, network-accessible storage, etc.

These machine-executable instructions may be stored on one or more machine-readable mediums, such as CD-ROMs or other type of optical disks, floppy diskettes, ROMs, RAMS, EPROMS, EEPROMs, magnetic or optical cards, flash memory, or other types of machine-readable mediums suitable for storing electronic instructions. Alternatively, the methods may be performed by a combination of hardware and software.

In another embodiment, a microprocessor may be a system or collection of processing hardware components, such as a microprocessor on a client device and a microprocessor on a server, a collection of devices with their respective microprocessor, or a shared or remote processing service (e.g., “cloud” based microprocessor). A system of microprocessors may comprise task-specific allocation of processing tasks and/or shared or distributed processing tasks. In yet another embodiment, a microprocessor may execute software to provide the services to emulate a different microprocessor or microprocessors. As a result, a first microprocessor, comprised of a first set of hardware components, may virtually provide the services of a second microprocessor whereby the hardware associated with the first microprocessor may operate using an instruction set associated with the second microprocessor.

While machine-executable instructions may be stored and executed locally to a particular machine (e.g., personal computer, mobile computing device, laptop, etc.), it should be appreciated that the storage of data and/or instructions and/or the execution of at least a portion of the instructions may be provided via connectivity to a remote data storage and/or processing device or collection of devices, commonly known as “the cloud,” but may include a public, private, dedicated, shared and/or other service bureau, computing service, and/or “server farm.”

Examples of the microprocessors as described herein may include, but are not limited to, at least one of Qualcomm® Snapdragon® 800 and 801, Qualcomm® Snapdragon® 610 and 615 with 4G LTE Integration and 64-bit computing, Apple® A7 microprocessor with 64-bit architecture, Apple® M7 motion co-microprocessors, Samsung® Exynos® series, the Intel® Core™ family of microprocessors, the Intel® Xeon® family of microprocessors, the Intel® Atom™ family of microprocessors, the Intel Itanium® family of microprocessors, Intel® Core® 15-4670K and i7-4770K 22 nm Haswell, Intel® Core® i5-3570K 22 nm Ivy Bridge, the AMD® FX™ family of microprocessors, AMD® FX-4300, FX-6300, and FX-8350 32 nm Vishera, AMD® Kaveri microprocessors, Texas Instruments® Jacinto C6000™ automotive infotainment microprocessors, Texas Instruments® OMAP™ automotive-grade mobile microprocessors, ARM® Cortex™-M microprocessors, ARM® Cortex-A and ARM926EJ-S™ microprocessors, other industry-equivalent microprocessors, and may perform computational functions using any known or future-developed standard, instruction set, libraries, and/or architecture.

Any of the steps, functions, and operations discussed herein can be performed continuously and automatically.

The exemplary systems and methods of this invention have been described in relation to communications systems and components and methods for monitoring, enhancing, and embellishing communications and messages. However, to avoid unnecessarily obscuring the present invention, the preceding description omits a number of known structures and devices. This omission is not to be construed as a limitation of the scope of the claimed invention. Specific details are set forth to provide an understanding of the present invention. It should, however, be appreciated that the present invention may be practiced in a variety of ways beyond the specific detail set forth herein.

Furthermore, while the exemplary embodiments illustrated herein show the various components of the system collocated, certain components of the system can be located remotely, at distant portions of a distributed network, such as a LAN and/or the Internet, or within a dedicated system. Thus, it should be appreciated, that the components or portions thereof (e.g., microprocessors, memory/storage, interfaces, etc.) of the system can be combined into one or more devices, such as a server, servers, computer, computing device, terminal, “cloud” or other distributed processing, or collocated on a particular node of a distributed network, such as an analog and/or digital telecommunications network, a packet-switched network, or a circuit-switched network. In another embodiment, the components may be physical or logically distributed across a plurality of components (e.g., a microprocessor may comprise a first microprocessor on one component and a second microprocessor on another component, each performing a portion of a shared task and/or an allocated task). It will be appreciated from the preceding description, and for reasons of computational efficiency, that the components of the system can be arranged at any location within a distributed network of components without affecting the operation of the system. For example, the various components can be located in a switch such as a PBX and media server, gateway, in one or more communications devices, at one or more users' premises, or some combination thereof. Similarly, one or more functional portions of the system could be distributed between a telecommunications device(s) and an associated computing device.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

Also, while the flowcharts have been discussed and illustrated in relation to a particular sequence of events, it should be appreciated that changes, additions, and omissions to this sequence can occur without materially affecting the operation of the invention.

A number of variations and modifications of the invention can be used. It would be possible to provide for some features of the invention without providing others.

In yet another embodiment, the systems and methods of this invention can be implemented in conjunction with a special purpose computer, a programmed microprocessor or microcontroller and peripheral integrated circuit element(s), an ASIC or other integrated circuit, a digital signal microprocessor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this invention. Exemplary hardware that can be used for the present invention includes computers, handheld devices, telephones (e.g., cellular, Internet enabled, digital, analog, hybrids, and others), and other hardware known in the art. Some of these devices include microprocessors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein as provided by one or more processing components.

In yet another embodiment, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this invention is dependent on the speed and/or efficiency requirements of the system, the particular function, and the particular software or hardware systems or microprocessor or microcomputer systems being utilized.

In yet another embodiment, the disclosed methods may be partially implemented in software that can be stored on a storage medium, executed on programmed general-purpose computer with the cooperation of a controller and memory, a special purpose computer, a microprocessor, or the like. In these instances, the systems and methods of this invention can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Embodiments herein comprising software are executed, or stored for subsequent execution, by one or more microprocessors and are executed as executable code. The executable code being selected to execute instructions that comprise the particular embodiment. The instructions executed being a constrained set of instructions selected from the discrete set of native instructions understood by the microprocessor and, prior to execution, committed to microprocessor-accessible memory. In another embodiment, human-readable “source code” software, prior to execution by the one or more microprocessors, is first converted to system software to comprise a platform (e.g., computer, microprocessor, database, etc.) specific set of instructions selected from the platform's native instruction set.

Although the present invention describes components and functions implemented in the embodiments with reference to particular standards and protocols, the invention is not limited to such standards and protocols. Other similar standards and protocols not mentioned herein are in existence and are considered to be included in the present invention. Moreover, the standards and protocols mentioned herein and other similar standards and protocols not mentioned herein are periodically superseded by faster or more effective equivalents having essentially the same functions. Such replacement standards and protocols having the same functions are considered equivalents included in the present invention.

The present invention, in various embodiments, configurations, and aspects, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving case, and/or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments, configurations, or aspects for the purpose of streamlining the disclosure. The features of the embodiments, configurations, or aspects of the invention may be combined in alternate embodiments, configurations, or aspects other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the invention.

Moreover, though the description of the invention has included description of one or more embodiments, configurations, or aspects and certain variations and modifications, other variations, combinations, and modifications are within the scope of the invention, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

What is claimed is:

1. A method of updating a model, the method comprising:
detecting a change in an output of the model responsive to providing a set of inputs to the model;
in response to detecting the change in the output, triggering a new model detection flag using a blockwise recursive partial least squares (RPLS) algorithm;
assessing a performance of a new model using a set of prediction metrics;
storing the new model in memory;
comparing a performance of the model using the set of inputs with the performance of the new model using the set of prediction metrics; and
based on the comparison and in response to the comparison indicating that the performance of the new model using the set of prediction metrics is improved relative to the performance of the model using the set of inputs, replacing the model with the new model in run-time.

2. The method of claim 1, wherein the memory comprises buffer memory.

3. The method of claim 2, further comprising:
validating the new model on buffer process data.

4. The method of claim 3, wherein the buffer process data comprises at least one of the following: a past data block, a present data block, and a data block to be received.

5. The method of claim 4, wherein a size of the buffer memory is selected to minimize an impact on data processing while validating the new model.

6. The method of claim 1, further comprising:
iteratively repeating the detecting, the triggering, the assessing, the storing, the comparing, and the replacing.

7. The method of claim 1, further comprising:
storing the model in memory with a data signature that is unique to the model; and
enabling retrieval of the model in real-time based on a detection of another data signature in process data having a predetermined similarity to the data signature of the model stored in memory.

8. The method of claim 1, further comprising:
maintaining a model library that includes the model and the new model, wherein the model library is indexed according to data signatures associated with each model in the model library.

9. The method of claim 1, further comprising:
determining an angle between weight matrices of a master model and weight matrices of the new model; and
correlating the angle to a measure of change between an input block and an output block of the master model and the new model.

10. A system, comprising:
a processor; and
a memory capable of storing data thereon that, when processed by the processor, cause the processor to:
detect a change in an output of a model responsive to providing a set of inputs to the model;
in response to detecting the change in the output, trigger a new model detection flag using a blockwise recursive partial least squares (RPLS) algorithm;
assess a performance of a new model using a set of prediction metrics;
store the new model in a buffer;
compare a performance of the model using the set of inputs with the performance of the new model using the set of prediction metrics; and
based on the comparison and in response to the comparison indicating that the performance of the new model using the set of prediction metrics is improved relative to the performance of the model using the set of inputs, replace the model with the new model in run-time.

11. The system of claim 10, wherein the data further enables the processor to:
validate the new model on buffer process data.

12. The system of claim 11, wherein the buffer process data comprises at least one of the following: a past data block, a present data block, and a data block to be received.

13. The system of claim 12, wherein a size of the buffer is selected to minimize an impact on data processing while validating the new model.

14. The system of claim 13, wherein the data further enables the processor to:
iteratively repeat the detecting, the triggering, the assessing, the storing, the comparing, and the replacing.

15. The system of claim 11, wherein the data further enables the processor to:
store the model in memory with a data signature that is unique to the model; and
enable retrieval of the model in real-time based on a detection of another data signature in process data having a predetermined similarity to the data signature of the model stored in the memory.

16. The system of claim 11, wherein the data further enables the processor to:
maintain a model library that includes the model and the new model, wherein the model library is indexed according to data signatures associated with each model in the model library.

17. The system of claim 11, wherein the data further enables the processor to:
determine an angle between weight matrices of a master model and weight matrices of the new model; and
correlate the angle to a measure of change between an input block and an output block of the master model and the new model.

18. A non-transitory computer-readable medium comprising processor-executable instructions stored thereon that enable a processor to:
detect a change in an output of a model responsive to providing a set of inputs to the model;
in response to detecting the change in the output, trigger a new model detection flag using a blockwise recursive partial least squares (RPLS) algorithm;
assess a performance of a new model using a set of prediction metrics;
store the new model in a buffer;
compare a performance of the model using the set of inputs with the performance of the new model using the set of prediction metrics; and
based on the comparison and in response to the comparison indicating that the performance of the new model using the set of prediction metrics is improved relative to the performance of the model using the set of inputs, replace the model with the new model in run-time.

19. The non-transitory computer-readable medium of claim 18, wherein the instructions further enable the processor to: validate the new model on buffer process data, wherein the buffer process data comprises at least one of the following: a past data block, a present data block, and a data block to be received.

20. The non-transitory computer-readable medium of claim 18, wherein the instructions further enable the processor to determine an angle between weight matrices of a master model and weight matrices of the new model, and correlate the angle to a measure of change between an input block and an output block of the master model and the new model.
